(19) United States
(12) Patent Application Publication
(10) Pub. No.: US 2013/0096595 A1
(43) Pub. Date: Apr. 18, 2013

(54) METHODS AND SYSTEMS FOR INDUCING HYPERTHERMIA

(71) Applicants: Gunnar Myhr, Oslo (NO); Bjorn A.J. Angelsen, Trondheim (NO)

(72) Inventors: Gunnar Myhr, Oslo (NO); Bjorn A.J. Angelsen, Trondheim (NO)

(21) Appl. No.: 13/641,520

(22) PCT Filed: Apr. 14, 2011
(86) PCT No.: PCT/GB2011/050746

§ 371 Date: Dec. 21, 2012

(30) Foreign Application Priority Data
Apr. 16, 2010 (GB) 1006446.7
Apr. 16, 2010 (US) 61324891

(51) Int. Cl. A61N 7/02

(52) U.S. Cl. CPC A61N 7/022
USPC 606/169

(57) ABSTRACT

A system and methods are provided and thrombi treatments in which hyperthermia is induced in an initial phase and cavitation and/or drug release are induced in a subsequent phase in a region of interest in a human or animal body. The system includes an energy transmitter having a variable intensity and/or a variable frequency; and a control unit arranged to control the energy transmitter to operate in at least two different modes. In one embodiment, the first mode is a mode of operation having a mechanical index below a threshold level for cavitation; and the second mode is a mode of operation having a mechanical index above a threshold level for cavitation. In another embodiment, the first mode induces hyperthermia below a temperature threshold for releasing an encapsulated agent and the second mode induces hyperthermia above the temperature threshold. The initial hyperthermia treatment enhances the effect of subsequent treatments.

The present invention relates to apparatuses and systems for cancer and thrombi treatment. More particularly the invention relates to the use of energy sources with variable intensities and/or frequencies for inducing hyperthermia and optionally also cavitation.

The present invention is a further development of the inventor(s) prior International Patent Application WO2006/129099 “Ultrasound treatment system” and Patent GB 2450249 “Magnetic resonance guided cancer treatment system”, in addition to the journal papers Technol Cancer Res Treat 2008; 5; 409-414, Med Hypotheses 2008; 70; 665-670 and Med Hypotheses 2007; 69; 1325-1333, authored by one of the inventors, which are hereby incorporated by reference.

Traditionally, the primary curative treatment for solid tumors has been surgery. Adjuvant or sequential therapy for cancer usually refers to surgery preceding or following chemotherapy and/or ionizing radiation treatment to decrease the risk of recurrence. Recent studies have determined that the absolute benefit for survival obtained with adjuvant therapy compared to control is still only approximately 6%, while 5-year survival benefit attributable solely to cytotoxic chemotherapy is approximately 2%.

Tumor hypoxia represents a primary therapeutic concern since it can reduce the effectiveness of drugs and radiotherapy. Well-oxygenated cells require one-third the dose of hypoxic cells to achieve a given level of cell killing. Multi-drug resistance (MDR) in cancer cells can also cause simultaneous resistance to anticancer drugs.

Generally, the most important physiological response to heat is blood flow. Mild hyperthermia with a temperature increase from 37 to 40 degrees C. has been shown to cause mean intra-tumor partial oxygen pressure, pO₂ to increase by 25% from, e.g. 16 to 20 mm Hg. Increased pO₂ enhances tumour radiosensitization. Subsequently, in one study, when ionizing radiation was applied at 18 Gy, regrowth delay increased by a factor of 1.7. Hyperthermia also causes the extravasation of liposome nanoparticles in different tumor regions. Experiments on murine mamma carcinoma 4T1 cell lines with rhodamine-labelled nanoparticles (D=100 nm) have shown that the relative particle density was 3 times higher in the tumor periphery than in the tumor center, with a temperature increase from 34 to 42 degrees C., after 1 h of hyperthermia. Ionizing radiation has also been shown to improve the distribution and uptake of liposomal doxorubicin (Caelyx) in human osterosarcoma xenografts, without the disintegration of the liposomes.

Blood clots (fibrin clots) are the clumps that result from coagulation of the blood. A blood clot that forms in a vessel or within the heart and remains there is called a thrombus. A thrombus that travels from the vessel or heart chamber where it formed to another location in the body is called an embolus, and the disorder, an embolism (for example, pulmonary embolism). Sometimes a piece of atherosclerotic plaque, small pieces of tumour, fat globules, air, amniotic fluid, or other materials can act in the same manner as an embolus. Thrombi and emboli can firmly attach to a blood vessel and partially or completely block the flow of blood in that vessel. This blockage deprives the tissues in that location of normal blood flow and oxygen. This is called ischemia and if not treated promptly, can result in damage or even death of the tissues (infarction and necrosis) in that area.

Deep venous thrombosis (DVT) refers to a blood clot embedded in one of the major deep veins of the lower legs, thighs, or pelvis. A clot blocks blood circulation through these veins, which carry blood from the lower body back to the heart. The blockage can cause pain, swelling, or warmth in the affected leg.

Blood clots in the veins can cause inflammation (irritation) called thrombophlebitis. The most worrisome complications of DVT occur when a clot breaks loose (or embolizes) and travels through the bloodstream and causes blockage of blood vessels (pulmonary arteries) in the lung. This can lead to severe difficulty in breathing and even death, depending on the degree of blockage.

In the United States, about 2 million people per year develop DVT. Most of them are aged 40 years or older. Statistics reveal that at least 200,000 patients die each year from blood clots in their lung.

Cavitation is the growth and collapse of naturally or artificially added microbubbles in a bodily fluid, and is dependent on the mechanical index, MI. MI=(P_{neg})/f^{1/2}), where P_{neg}=maximum negative pressure (in MPa) and f=frequency (in MHz). The cavitational activity is thus inversely related to frequency.

Ultrasound energy absorption (attenuation), is a function of frequency. I(r)=I₀ exp [−μ(f)r] where I(r)=intensity at tissue depth r, I₀=output intensity in non absorbing material and μ(f)=intensity-absorption coefficient, which is a function of frequency and type of tissue.

Energy absorption increases with increasing frequency. The challenge is to reach a well defined volume within the (human or animal) patient, (i.e. a region of interest (ROI)) with a high intensity of acoustic energy at a low frequency, enabling to limit the exposure to a relatively small region of interest, and at the same time minimizing the acoustic exposure to surrounding tissues.

Synergistic effects of low frequency ultrasound exposure in combination with liposomally encapsulated doxorubicin (Caelyx), subjected to very sub optimal conditions have been demonstrated (Cancer Lett. 2006; 232; 206-213). Balb/c nude mice were inoculated with a WiDr (human colon cancer) tumor cell line at various concentrations. Tumor growth inhibition was delayed by 30-40%, based on 2 treatments under non-hyperthermic conditions. The mouse tumors were cooled to 24 degrees C. to exclude any hyperthermic effects (from about 30 degrees C.). This probably caused the tumor vasculature to contract, restricting both blood and drug supply. The drug was administered one hour before treatment. It is known that peak drug concentrations occur between 48 and 72 hours. Synergistic ultrasound mediated drug release has also been verified by others' researches.

In one study, echogenic liposomes (ELIP) were used as vehicles for delivering oligonucleotides (ODN). Application of ultrasound (1 MHz continuous wave, 0.26 MPa peak-to-peak pressure amplitude, 60 s) triggered 41.6+/−4.3% release of ODN from ODN-containing ELIP.

ELIP have also been developed as ultrasound-triggered targeted drug or gene delivery vehicles. These vesicles have the potential to be used for ultrasound-enhanced thrombolysis in the treatment of acute ischemic stroke, myocardial infarction, deep vein thrombosis or pulmonary embolus. Tissue plasminogen activator (tPA) and tPA incorporated ELIP, are labeled T-ELIP. The conclusions are that T-ELIP are robust and echogenic during continuous fundamental 6.9 MHz B-mode imaging at a low exposure output level (600 kPa). Furthermore, a therapeutic concentration of rt-PA can be released by fragmenting the T-ELIP with pulsed 6.0 MHz color Doppler ultrasound above the rapid fragmentation threshold (1.59 MPa).

In another study, doxorubicin and microbubble loaded liposomes killed at least two times more melanoma cells after exposure to ultrasound.

In Ultrasonics. 2006 December; 45 (1-4); 133-45, changes in membrane permeation (leakage mimicking drug release) were investigated in vitro during exposure to ultrasound applied in two frequency ranges: “conventional” (1 MHz and 1.6 MHz) therapeutic ultrasound range and low (20 kHz) frequency range. Phospholipids vesicles were used as controllable biological membrane models. The membrane properties were modified by changes in vesicle dimensions and incorporation of poly(ethylene glycol) i.e. PEGylated lipids. Egg phosphatidylcholine vesicles with 5 mol % PEG were prepared with sizes ranging from 100 nm to 1 micrometer. Leakage was quantified in terms of temporal fluorescence intensity changes observed during controlled ultrasound. Custom-built transducers operating at frequencies of 1.6 MHz (focused) and 1.0 MHz (unfocused) were used, the intensities, I(spta) were 46.9 W/cm² and 3.0 W/cm², respectively. A commercial 20 kHz, point-source, continuous wave transducer with an intensity, I(spta) of 0.13 W/cm² was also used for comparative purposes. Whereas complete leakage was obtained for all vesicle sizes at 20 kHz, no leakage was observed for vesicles smaller than 100 nm in diameter at 1.6 or 1.0 MHz. However, introducing leakage at the higher frequencies became feasible when larger (greater than 300 nm) vesicles were used, and the extent of leakage correlated well with vesicle sizes between 100 nm and 1 micrometer.

In J Nanosci Nanotechnol. 2007 March; 7 (3); 1028-1033, polymer (Pluronic 105) encapsulated doxurubicin was released at intensities above a threshold values of 0.4 W/cm² at ultrasound frequency of 70 kHz. The Mechanical Index corresponding to 0.4 W/cm² is 0.4.

In Clin Cancer Res. 2007 May 1; 13 (9); 2722-7, comparisons in vitro and in vivo were carried out between non-thermosensitive liposomes (NTSL) and low temperature-sensitive liposomes (LTSL). Liposomes were incubated in vitro over a range of temperatures and durations, and the amount of doxorubicin released was measured. For in vivo experiments, liposomes and free doxorubicin were injected intravenously in mice followed by pulsed-HIFU exposures in sarcomatoid carcinoma murine adenocarcinoma tumors at 0 and 24 h after administration. Combinations of the exposures and drug formulations were evaluated for doxorubicin concentration and growth inhibition in the tumours. In vitro incubations simulating the pulsed-HIFU (High Intensity Focused Ultrasound) thermal dose (42 degrees C. for 2 min) triggered release of 50% of doxorubicin from the LTSLs; however, no detectable release from the NTSLs was observed. Similarly, in vivo experiments showed that pulsed-HIFU exposures combined with the LTSLs resulted in more rapid delivery of doxorubicin as well as significantly higher intratumoral concentration when compared with LTSLs alone or NTSLs, with or without exposures.

In Ultrason Sonochem. 2005 August; 12 (6); 489-93, the effect of ultrasound on liposome-mediated transfection was investigated. Three types of liposomes containing O,O′-ditetradecanoyl-N-(alpha-trimethylammonioacetyl) diethanolamine chloride, dioleoylphosphatidylethanolamine, and/or cholesterol at varying ratios, were used in this study. HeLa cells were treated with liposome-DNA complexes containing luciferase genes for 2 h before sonication. Optimal ultrasound condition for the enhancement was determined to be 0.5 W/cm², 1 MHz continuous wave for 1 min and was above threshold for inertial cavitation based on EPR (Electron Paramagnetic Resonance) detection of free radicals.

In Technol Cancer Res Treat. 2007 February; 6 (1); 49-56, a study of steady state acoustic release of Doxorubicin from Pluronic P105 micelles using Artificial Neural Networks (ANN) was done. The model showed that drug release was most efficient at lower frequencies. The analysis also demonstrated that release increases as the power density increases. Sensitivity plots of ultrasound intensity revealed a drug release threshold of 0.015 W/cm² and 0.38 W/cm² at 20 kHz and 70 kHz, respectively. The presence of a power density threshold provides strong evidence that cavitation plays an important role in acoustically activated drug release from polymeric micelles. Based on the developed model, doxorubicin release is not a strong function of temperature, suggesting that thermal effects do not play a major role in the physical mechanism involved.

In Cancer Chemother Pharmacol. 2009 August; 64 (3); 593-600, a study employed ultrasound of two different frequencies (20, 476 kHz) and two pulse intensities, but identical mechanical indices and temporal average intensities. Ultrasound was applied weekly for 15 min to one of two bilateral leg tumors (DHD/K12/TRb colorectal epithelial cell line) in a rat model immediately after intravenous injection of micelle-encapsulated doxorubicin. This therapy was applied weekly for 6 weeks. Results showed that tumors treated with drug and ultrasound displayed, on average, slower growth rates than non-insonated tumors. However, comparison between tumors that received 20 or 476-kHz ultrasound treatments showed no statistical difference in tumor growth rate.

In J Control Release. 2009 Aug. 19; 138 (1); 45-8 a custom-made ultrasound exposure chamber was used with fluorescence detection to measure the long-term fluorescence emissions of doxorubicin after 2 h of exposure to two ultrasound frequencies, 70 and 476 kHz, at a mechanical index of 0.9. Fluorescence measurements were then used to deduce the degradation kinetics of stabilized Pluronic micelles during 24 h following exposure to ultrasound. Results showed that ultrasound does disrupt the covalent network of the stabilized micelles, but the time constant of network degradation is very long compared to the time constant pertaining to drug release from micelles. Experiments also showed no significant difference in degradation rates when employing the two frequencies in question at the same mechanical index.

In Tree Physiology 2007; 27; 969-976, T1-weighted spin-echo sequences were used with a repetition time (TR) of 500 ms and an echo time (TE) of 22 ms to obtain 2-D images of the water content in a transverse section of pine stem. The values of TR and TE were determined in preliminary experiments to produce images of cavitation in xylem with the highest contrast. Proton density sequences with longer TRs are usual when imaging water in living organisms, however, not only water in conducting xylem but also resinous materials in embolized xylem produce strong signals in the proton density sequence because resins contain protons. T1-weighted image with a short TR was most suitable for differentiating between functional and embolized xylem in the system.

Ultrasound can also be used to detect cavitation and ultrasound (radiation force) induced streaming. Work is also in progress on using ultrasound to detect temperature.

Acoustic streaming is a bulk flow caused by attenuation of an acoustic wave propagating in a medium (radiation force). Measurements of acoustic streaming can provide information on the cavitation field. MRI methods have been applied to studies of acoustic streaming in a cavitating fluid. In Ultrasound in Medicine & Biology 2004; 30 (9); 1209-1215, both temperature and cavitation were monitored by MRI.

To measure the hemodynamic response related to neural activity in the brain by MRI, is well known as functional MRI (fMRI). Oxygen consumption or partial oxygen pressure (pO₂) and/or blood flow effects oxyhemoglobin and deoxyhemoglobin, called blood oxygen level-dependent (BOLD), and can be monitored in particular by T₂^{*}-weighted MRI images, are also well described in the literature, e.g. NMR Biomed. 2006 February; 19 (1); 84-9.

According to a first aspect of the invention, there is provided a system for inducing hyperthermia and cavitation in a region of interest in a human or animal body comprising: an energy transmitter having a variable intensity and/or a variable frequency; and a control unit arranged to control the energy transmitter to operate in at least two different modes, wherein a first mode is a mode of operation having a mechanical index below a threshold for cavitation; and wherein a second mode is a mode of operation having a mechanical index above a threshold for cavitation.

According to the invention, existing technology available for tissue heating or destruction can be adapted for an additional application, namely to supply different energy levels for a different treatment. As discussed above, application of heat leads to increased blood flow, increased oxygenation and increased transport of therapeutic agents (if present). By enabling a hyperthermia treatment alongside a cavitation based treatment, using the same device with a variable intensity and/or frequency, a simple system is provided for applying synergistic treatments. The cavitation based treatment may be just cavitation of naturally occurring microbubbles. However, in other preferred forms the treatment may involve the combination of cavitation of naturally occurring microbubbles with a further therapeutic agent via sonoporation or it may involve the cavitation of added microbubbles or the decapsulation of encapsulated agents.

In these contexts, a therapeutic agent may include one or more of a drug, gas or fluid filled bubbles of nano or micron size, liposomally, protein or polymer encapsulated agents, or combinations thereof.

Added microbubbles may be in the form of encapsulated gas bubbles. In one preferred embodiment, this may be in the form of nanoparticles filled with perfluorcarbon that evaporates at around 40 degrees centigrade and thereby produce micro gas bubbles. Perfluorcarbons also absorb oxygen to a high degree (e.g. about 40 volume %). Therefore encapsulated oxygen saturated perfluorcarbon can be used to increase oxygen transport to the region of interest where it is released through evaporation induced by hyperthermia and subsequently by cavitation of the encapsulating vesicle. Sonoporation of barriers and membranes can further help transport of oxygen across these barriers and cell membranes.

Preferably the energy transmitter comprises an ultrasound transmitter. The ultrasound transmitter may be a single transducer or an array of transducers. Single transducers may be focused by shaping the transducer. Arrays of transducers allow beam forming and focusing techniques to be used, e.g. for electronically steered targeting of a defined ROI.

Preferably, the energy transmitter comprises a high intensity focused ultrasound transmitter, more preferably with electronically steered focus depth and direction.

In the past, frequencies for tissue heating and/or destruction have typically been high (e.g. 1 MHz to 5 MHz) in order to ensure sufficient energy deposition in the region of interest, whereas frequencies for inducing cavitation have typically been low (e.g. 20 kHz to about 500 kHz) as cavitation is more achievable at low frequencies.

According to preferred embodiments of the present invention, a single frequency band transmitter can be used to induce both hyperthermia and cavitation provided that the mechanical index is controlled appropriately. This allows readily available (off-the-shelf) high intensity focused ultrasound (HIFU) units to be used in combinatorial treatments. For example, the system can be used to provide simultaneous or sequential hyperthermia and cavitation based treatments. The system therefore provides a more cost effective system as only a single transmitter (transducer) is required where previously two transmitters of different frequencies would have been required.

It is possible to use the same frequency for heating and inducing cavitation, but this is not an optimal arrangement. If the same frequency is used for first heating with a Mechanical Index below a threshold value, and then one increases the Mechanical Index to induce cavitation (and possibly particle cracking), one could potentially provide overheating with damage of the surrounding tissue. This may be acceptable in some circumstances, especially where the application time of the higher mechanical index phase can be kept relatively short. This arrangement is particularly advantageous as the readily available HIFU units are designed for operation at a single frequency.

The ultrasound frequency for heating is determined by how to deploy the most absorbed power at the ROI depth. Ultrasound absorption increases with frequency, but for a given depth one must still ensure that the beam intensity is adequately high so that there is some power to absorb. Due to absorption, the power available for heating decreases with depth and increasing frequency. For maximal heating at a given depth, there is hence an optimal frequency, that decreases approximately inversely with increasing depth.

In preferred embodiments, the ultrasound transmitter is a single frequency band transmitter. Such ultrasound transducers are designed to operate at a single resonant frequency which depends on the transducer properties (e.g. material, thickness). Previous applications have all been based around that single frequency. However, with minor changes to the control electronics/software, the transducer can be driven at a different off-resonant frequency with a reduction in intensity. Using this technique, ultrasound transducers can typically operate with a relative bandwidth of around 50-60%, e.g. within a frequency range 30% either side of the centre frequency. With this arrangement, the present invention allows the two modes of operation to be performed at different frequencies with the same transducer. By applying a lower frequency to the cavitation mode, tissue is less likely to suffer overheating during this phase of treatment.

Preferably therefore, in the first mode of operation the ultrasound transmitter is arranged to operate at a high frequency above the centre frequency of the frequency band and in the second mode of operation the ultrasound transmitter is arranged to operate at a low frequency below the centre frequency of the frequency band. More preferably the low frequency is up to 30% lower than the centre frequency and wherein the high frequency is up to 30% higher than the centre frequency. In order to obtain a sufficient frequency separation between the two modes, preferably the low frequency is at least 5% lower than the centre frequency and wherein the high frequency is at least 5% higher than the centre frequency.

In preferred embodiments, the ultrasound transmitter is arranged to operate with a centre frequency in the range of 0.3 to 30 MHz. Various frequencies are used for different purposes. For example, frequencies up to 30 MHz can be used for skin cancers, whereas 0.3 MHz can be used for high MI cavitation at deeper depths such as for the liver and kidneys. Breast and prostate treatments may use frequencies of 5 to 10 MHz. This frequency range is more typically associated with hyperthermia treatments and will therefore deposit a sufficient amount of energy in the tissue in the region of interest to cause hyperthermia. In more preferred embodiments, the ultrasound transmitter is arranged of operate with a centre frequency in the range of 1 MHz to 5 MHz, more preferably still, in the range of 1.2 to 1.7 MHz. Most commercially available HIFU units operate in this range and therefore the invention can be put into practice with the equipment which is readily available.

In alternative embodiments, dual band ultrasound transducers can be used. Such transducers can be driven in either of two different frequency bands and can provide a greater separation between the frequencies used in the two modes of operation. Although such dual band transducers can provide greater flexibility, there are no existing dual band ultrasound transmitters readily available in the marketplace, thereby adding to the cost of implementation.

Therefore in preferred embodiments, the ultrasound transmitter has at least two frequency bands and the transmitter is arranged to operate in the higher frequency band in the first mode of operation and the transmitter is arranged to operate in the lower frequency band in the second mode of operation.

Preferably the first mode of operation is suitable for inducing hyperthermia in the region of interest, but is not suitable for inducing cavitation in the region of interest. With this arrangement, the hyperthermia phase can be used to increase the oxygenation and/or levels of therapeutic agents in the region of interest before the more active stage of treatment commences. For example, the hyperthermia phase can be used to increase the oxygenation and the concentration of encapsulated drugs or microbubbles without causing rupture of the encapsulating vesicles. By causing a greater concentration to build up in this way, the second phase of treatment (i.e. the second mode of operation) becomes more effective. Also the greater time period of the initial hyperthermia treatment can be used to cause therapeutic agents to penetrate deeper into the hypoxic fractions of tumours, thereby increasing the efficacy of the treatment as a whole.

Although the second mode of treatment may be simply to induce cavitation of naturally occurring microbubbles, the second mode of operation is preferably suitable for releasing an encapsulated therapeutic agent. This encapsulated agent may be encapsulated gas microbubbles, perfluorcarbons or other chemotherapeutic agents as discussed above. Therefore, in preferred embodiments the second mode of operation is suitable for inducing cavitation. Cavitation can increase uptake of therapeutic agents by the tissue in the region of interest through sonoporation (transport across membranes like cell membranes and blood-brain barrier or other barriers). Cavitation is also a mechanism behind release of encapsulated therapeutic agents, i.e. it is a mechanism for rupturing the vesicles. Therefore in preferred embodiments the second mode of operation is suitable for rupturing the vesicles of an encapsulated therapeutic agent. As discussed above, many therapeutic agents are more effective in regions of increased oxygenation. Therefore the combination of increased oxygenation through hyperthermia with rupturing of encapsulated therapeutic agents is synergistically beneficial.

In some preferred embodiments, the transmitter is further arranged to operate in a third mode of operation having a mechanical index above the threshold level for cavitation and being suitable for inducing cavitation of microbubbles. As discussed above, cavitation can increase drug uptake via sonoporation. Therefore, a treatment phase of inducing cavitation can be further useful after a decapsulating phase.

According to another aspect, the invention provides a system for inducing hyperthermia and encapsulated agent release in a region of interest in a human or animal body comprising: an energy transmitter having a variable intensity and/or a variable frequency; and a control unit arranged to control the energy transmitter to operate in at least two different modes, wherein a first mode is a mode of operation having an energy level below a threshold temperature level; and wherein a second mode is a mode of operation having an energy level above a threshold temperature level.

It will be appreciated that this aspect of the invention provides an alternative solution to the problem of inducing hyperthermia and encapsulated agent release via a single transmitter. As discussed above, thermosensitive liposomes can be used for encapsulating therapeutic agents. These thermosensitive vesicles are preferably arranged to break down at a temperature higher than the hyperthermia phase of the treatment. Previous uses of thermosensitive liposomes have not involved hyperthermia in the treatment. Heat has only been applied when it is desired to break the vesicles. However, according to this invention, the hyperthermia phase of the treatment causes the encapsulated agent to penetrate deeper into the region of interest and in greater concentrations. This increases the effectiveness of the agent when it is released in the second treatment phase.

It will be appreciated that many different heat sources could be used in this aspect of the invention. In some preferred embodiments, the energy transmitter comprises an electromagnetic energy transmitter. Although in some instances it may be desirable to use frequencies up to Terrahertz levels, preferably the electromagnetic energy transmitter is arranged to operate at a frequency between 100 MHz and 4 GHz.

An other embodiments, the energy transmitter comprises an ultrasound transmitter. In contrast with the above first aspect of the invention, the mechanical index is not the key control feature in this aspect, but rather than rate of energy deposition must be controlled in order to control the temperature induced in the region of interest.

As with the first aspect, in preferred embodiments, the first mode of operation is suitable for inducing hyperthermia, but is not suitable for releasing an encapsulated agent, and the second mode of operation is suitable for rupturing the vesicles of an encapsulated agent.

The choice of encapsulated agent may be any of those described above in relation to the first aspect.

As discussed above, the benefits of inducing hyperthermia include increased blood flow with a corresponding increase in tissue oxygenation and an increase in transport of therapeutic agents into the region of interest (if used). Increased oxygenation and increased transport of therapeutic agents can in themselves be sufficient treatment. However these benefits are greatly increased when combined with cavitation or EM heat-induced breakage.

It is therefore possible to target the release of the therapeutic agent, using the focused ultrasonic or EM radio wave energy, at precisely the location where treatment is required. In this specification, the agent may be a drug or it may be a gas, fluid or combinations thereof. In the case of a gas, the collapse of the bubble or vesicle releases energy which is used to provide a treatment e.g. to have a therapeutic effect on a blood clot. In the case of a drug, this may be encapsulated in the interior of the “capsules” or attached to or incorporated in the membranes forming the capsule walls.

Typical hyperthermia levels in a human patient involve a temperature raise from 37 degrees C. to around 40 to 43 degrees C. Such temperatures are non-destructive in contrast to other heat related treatments (ablation treatments) which heat the tissue to much higher temperatures, e.g. greater than 50 degrees C. in order to cause tissue necrosis.

Preferably the energy transmitter comprises an ultrasound transmitter and wherein in the second mode of operation the ultrasound transmitter is operated at a subharmonic frequency. Although the ultrasound transmitter can most readily be operated at its main (fundamental) frequency, it is also possible to run such units at subharmonic frequencies. In particular, the transmitter can be operated at half the fundamental frequency. Although transmission at subharmonic frequencies can only be done at lower intensities, the frequency drop still leads to a gain in mechanical index. Therefore with this arrangement cavitation and/or encapsulated particle cracking can be achieved at lower intensities which means less heat is applied to the region of interest during this mode of operation.

Although the system could simply be used for two individual treatment types, the control unit is preferably further arranged to switch the energy transmitter from the first mode to the second mode. This allows the system to conduct sequential therapies, one without inducing cavitation and a subsequent one in which cavitation is induced. If encapsulated therapeutic agents are also present, the control unit can thus be arranged to begin switch modes in order to crack the encapsulating vesicles.

In preferred embodiments, the system further comprises a monitoring unit arranged to monitor at least one parameter related to oxygenation in the region of interest. By monitoring the oxygenation level in the region of interest, the effectiveness of the treatment can be monitored. As described above, oxygenation results from increased blood flow which in turn is caused by hyperthermia in the region of interest. Therefore monitoring the oxygenation level gives an indication of the success of a hyperthermia treatment. The monitoring unit may simply monitor an overall oxygenation level. However, preferably the monitoring unit is arranged to monitor the or each parameter spatially in and around the region of interest. By providing spatial indications of oxygenation, the success or otherwise of the treatment in areas in and around the region of interest can be monitored. Thus it can be determined if sufficient oxygenation has been achieved throughout the whole region of interest or if there are areas not yet sufficiently oxygenated. Further, such data can be used as an indication of hyperthermia being induced outside the region of interest. For certain very localized treatments it may be desirable to avoid excess heating outside the region of interest.

The monitoring unit may be any diagnostic medical imaging device, including XRay, MR Imaging, Computer Tomograph, Positron Emission Tomograph, Ultrasound Imager. In addition to tomography, full 3D imaging and volume imaging are also useful However, in preferred embodiments, the monitoring unit is a Magnetic Resonance Imaging (MRI) unit arranged to monitor at least one of partial oxygen pressure, partial carbon dioxide pressure, acidity and temperature in the region of interest. These parameters are all related to the oxygenation of the tissue and serve as good treatment indicators. The MRI unit may additionally be arranged to monitor temperature.

In other embodiments where ultrasound is used, the ultrasound unit may be used to monitor temperature.

Preferably the monitoring unit is arranged to supply data to the control unit and the control unit is arranged to switch the energy transmitter from the first mode to the second mode based on said data. As the MRI unit monitors oxygenation levels in the region of interest and certain therapeutic agents are more effective at higher oxygenation levels, the MRI unit can be arranged to ensure that a certain level of oxygenation is reached before releasing therapeutic agents from encapsulated vesicles. In this way the maximum benefit of the agents can be gained without increasing toxicity to the patient. There may be one or more oxygenation levels within the region of interest and the monitoring unit may be arranged to monitor one or more of these levels simultaneously or sequentially. Preferably therefore the control unit is arranged to switch the energy transmitter from the first mode to the second mode when the data indicates that at least one level of oxygenation of the region of interest has reached a threshold level.

It will be appreciated that oxygenation and certain chemotherapeutic drugs act as radiation sensitizers and therefore in preferred embodiments, radiation may also be applied after the hyperthermia treatment, either simultaneously or sequentially with release of therapeutic agents. This high spatial and temporal control of the radiosensitization achieves a more effective treatment to the patient with reduced toxicity outside the region of interest.

Preferably the monitoring unit is further arranged to monitor cavitation levels within the region of interest. This may be in the form of monitoring acoustic streaming, stable and/or inertial (transient) cavitation. In this document the terms acoustic streaming, stable and/or inertial (transient) cavitation are collectively called or termed cavitation.

Cavitation of naturally occurring or added microbubbles (e.g., but not limited to, liposomally or polymer encapsulated microbubbles) can increase the rate of uptake of (chemotherapeutic) agents administered to the region of interest. The effect of the cavitation can be calculated and therefore, by monitoring cavitation, the amount of drug uptake can be deduced. This data can be used simply as a quality control or it can be used as feedback for real time control of the treatment.

The information about the desired location where the energy is to be focused (i.e. the region of interest) may be provided by an appropriate diagnostic tool, e.g. an MRI unit or another imaging unit as discussed above. This tool is preferably used to determine the desired location with respect to a reference point in space, such as with respect to a fixed table or frame, or with respect to a reference point on the patient, and desired locations of tumors or thrombi may be mapped.

In preferred embodiments, using a digital diagnostic imaging device like X-ray, Computer Tomograph, Magnetic Resonance Imaging, Positron Emission Tomograph, ultrasound imaging and the like, stereometric coordinates to one or several regions of interest (tumors or blood clots) are established. The stereometric coordinates of the regions of interest are recorded by the control means. In preferred embodiments, the apparatus of the invention is provided in combination with a diagnostic unit for determining the information about the desired location.

The energy transmitting unit may be placed on a robotic arm which can be manually, electronically, hydraulically and/or pneumatically controlled. In the case of automatic control, the information about the desired location where the energy is to be focused (i.e. the region of interest), may be sufficient to carry out a predetermined treatment programme. For example, for a relatively small tumor or thrombus, a single focal point may be used so that the energy interacts with a therapeutic agent at that point. Alternatively, if a larger region of interest is mapped, the control means may cause a series of transmissions at different points throughout the region, following a predetermined pattern. The control unit preferably has also the capability of optimizing the position of the energy transmitting unit with respect to minimizing attenuation due to energy losses caused by bone, certain organs (e.g. lungs) natural cavities within the patient, and the like. The means of optimizing the position can be based on empirical values in relation to position data, and/or input from diagnostic and/or therapeutic devices.

The invention also extends to the use of the systems described above for the treatment of cancer or in treatment of one or more thrombi.

According to a further aspect, the invention provides a method of inducing hyperthermia and cavitation in a region of interest in a human or animal body, comprising: operating an energy transmitter which has a variable intensity and/or a variable frequency in a first mode of operation having a mechanical index below a threshold level for cavitation, and operating the energy transmitter in a second mode of operation having a mechanical index above a threshold level for cavitation.

According to a further aspect, the invention provides a method of inducing hyperthermia and encapsulated agent release in a region of interest in a human or animal body comprising: operating an energy transmitter which has a variable intensity and/or a variable frequency in a first mode of operation having an energy level below a threshold temperature level; and operating the energy transmitter in a second mode of operation having an energy level above a threshold temperature level.

Any of the preferred features described above in relation to the system, also apply to methods of treatment. The methods of treatment may be used for treatment of cancer or thrombi.

The theory behind the relationship between particle cracking and the mechanical index of applied ultrasound energy is given in the appendix to this description.

Preferred embodiments of the invention will now be described, by way of example only, and with reference to the accompanying drawings in which:

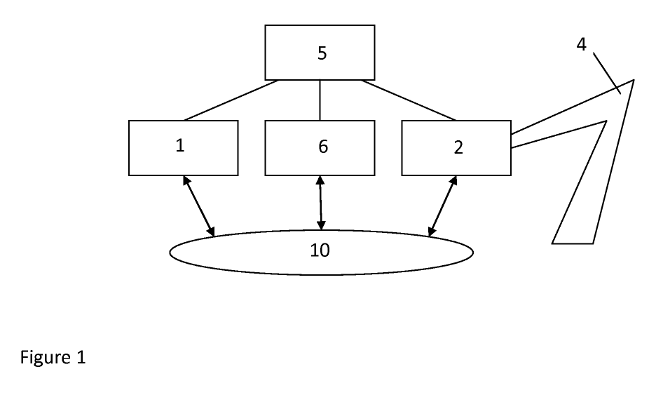

FIG. 1 shows a system according to the invention.

FIG. 1 shows one setup with reference to which a number of preferred embodiments of the invention will be described.

FIG. 1 shows a region of interest 10. An energy transmitting device 2 is arranged to direct energy at the ROI 10. The transmitting device 2 is mounted on a robotic arm 4. A monitoring unit 1 is arranged to monitor the ROI 10, e.g. by measuring various parameters in and around the ROI 10. A further treatment modality 6 is arranged to provide further treatment (e.g. chemotherapy or ionizing radiation) to the ROI 10. A control unit 5 is arranged to receive data from and transmit control signals to each of the energy transmitting unit 2, the monitoring unit 1, the robotic arm 4 and the further treatment modality 6.

It will be appreciated that the control unit may be a separate unit, or it may be integrated with any one of the other units. Additionally, the various elements depicted separately in FIG. 1 may be combined, for example a combined MRI and ultrasound unit could serve as both the monitoring unit and the energy transmitting device.

In one embodiment, a commercially available MRI unit 1 and a compatible HIFU unit 2 are provided. The HIFU unit 2 may have a single or multiple transducers or arrays of transducers 3. Transducers 3 may be mounted on one or several robotic arms 4 enabling transmission of ultrasound into a region of interest (ROI) at frequencies in the 20 kHz to 10 MHz range, most preferably in the 100 kHz to 3 MHz range. Transmission may be either continuous or pulsed transmission. The HIFU unit 2 can transmit at one or several frequencies, simultaneously, for example by transmitting at harmonic or subharmonic frequencies. The HIFU unit 2 can transmit at variable energy intensities, continuously or pulsed. Continuous acoustic intensities within tissues can vary in the range of 0 to 1000 W/cm², preferably in the 0 to 100 W/cm² range and they may cause a Mechanical Index (MI) at the ROI in the range of 0 to 10, preferably in the 0.1 to 6 range. Pulsed intensities can be up to 10000 W/cm² with a corresponding MI>100.

In another embodiment, the energy source 2 is an electromagnetic radiation source. In one preferred embodiment the electromagnetic radiation source is arranged to operate in the frequency range 1 to 100 MHz. In another embodiment the electromagnetic radiation source is arranged to operate in the frequency range 100 MHz to 4 GHz, with corresponding energy intensities to cause tissue temperatures in the 37 degrees C. to 100 degrees C. range, preferably in the 37 degrees C. to 46 degrees C. range.

The control unit 5 shown in FIG. 1 may comprise algorithms for providing energy levels below cavitational threshold levels, inducing hyperthermia in the 37 degrees C. to 46 degrees

C. range, preferably in the 40 degrees C. to 43 degrees C. range, at frequencies in the 20 kHz to 10 MHz range, preferably in the 1 MHz to 3 MHz range. The frequencies may be selected in order to cause the delivery of therapeutic agents into hypoxic fractions of tumours or into thrombi. The frequencies will depend on the tissue type, depth of the region of interest and, if used, the size and type of encapsulated therapeutic agents. In other embodiments, there may be no therapeutic agents, the treatment relying on cavitation alone. In other embodiments cavitation may be combined with non-encapsulated therapeutic agents to cause increased uptake of the agent by the target cells via sonoporation.

Therefore, subsequently and/or concurrently, with or without encapsulated agents, algorithms can provide energy levels above cavitational threshold levels, inducing energy levels associated with, but not limited to a mechanical index, MI>1, with, if ultrasound is applied, frequencies in the 20 kHz to 10 MHz range, preferably in the 0.1 MHz to 3MHz range.

In another embodiment, a commercially available MRI unit 1 may have an added or a built-in HIFU unit 2. Algorithms may be programmed into a computer (which may be built in or separate), causing the transducer(s) or array(s) to provide variable acoustic energies and frequencies. Further algorithms may be programmed into the computer for converting the MRI data into temperature measurements (MRI thermometry), pO₂, pCO₂ and/or pH. Thus, the system as a whole is arranged to execute hyperthermia in the region of interest 10, and to cause the selective release of encapsulated agents into the ROI 10, in real time.

Encapsulated agents may be supplied to the ROI 10 via the further treatment modality 6. Encapsulated agents may be supplied in any conventional way, e.g. orally or by injection.

Real time MRI monitoring facilitates approximate real time concomitant treatment options, including various combinations of drug therapy, hyperthermia, ionizing radiation, ablation and other treatment options, before or after surgery, with optimization capabilities.

Additionally, the MRI machine (monitoring unit 1) enables spatial monitoring and mapping of the region of interest, i.e. providing maps or gradients of pO₂, temperature, pH and/or CO₂ in a region of interest. The MRI machine can also monitor as a function of time by taking repeated measurements.

In these preferred embodiments, the ROI 10 is preferably modelled (e.g. mapped) before treatment commences. By modelling the ROI 10 (e.g. a tumor or a thrombus) before treatment, i.e. spatially mapping tissue in the region of interest (which can be done via a variety of techniques including MRI and CT scans) and mapping the location of the ROI with respect to reference points on the subject, it is possible subsequently to determine the levels of hyperthermia and pO₂, pH and/or CO₂ in relation to the position of the ROI, i.e. the position within the body. This data can be used either to correct the focus of the energy source which is applying the hyperthermia (e.g. to maintain accurate targeting of the region of interest) and/or to control the directionality of the further treatment modality (e.g. to control the direction and/or focus of applied radiation and/or applied ultrasound) to maximise the treatment effectiveness. Other factors, such as timing, intensity, fractionation and overall treatment time, i.e. total energy applied, can also be calculated more accurately using modelling of the region of interest.

In another embodiment, the system includes a computer (CPU) which is arranged to execute algorithms which provide temperature data in the 30-100 degrees C. range and partial oxygen pressure data in the range 0 mm Hg to 2000 mm Hg, from measurements of various parameters (e.g. T1, T2, PRF, Diffusion) by an MR unit in a defined volume within a living creature. The computer can also provide data on pH and CO₂. The computer/CPU can be an integral part of the MR unit (MR units typically include substantial computing power for complex processing and analysing of large quantities of data to produce MR images) or it can be part of a separate computation unit connected to the MR unit and receiving data from the MR unit. The computer/CPU can control the energy source, the position of the energy source(s) relative (or absolute) to the patient and the positioning of a robotic arm, in real time, the MR unit and/or the other treatment modalities.

In this embodiment, the computer is programmed with software algorithms for converting measured parameter data from the MR unit into calculated pO₂, temperature, pH and/or pCO₂ data. With sufficient computing power, these calculations can be carried out in real time. The computer uses this calculated data for quality control/feedback relating to the treatment. By comparing the calculated data with prestored mapping data of the ROI (which has been previously obtained through further MR or CT scans), the computer can determine both spatially and temporally the effectiveness of the treatment. For example, the computer can determine if the applied hyperthermia is sufficiently coincident with the region of interest and it can evaluate how long it takes for the hyperthermia to reach a desired level. The computer can use this analysis for feedback and control of the energy source to correct the focus, direction and/or intensity of the applied hyperthermia. The computer can also use the analysis for feedback and control of other applied treatment modalities such as radiotherapy and/or chemotherapy. For example, the computer can spatially and temporally monitor the pO₂ level within the region of interest and can use that data to begin application of radiotherapy or chemotherapy when a given pO₂ level has been reached, e.g. when the tissue has reached a state of being more receptive to those treatments by virtue of increased oxygenation and increased drug transport to the region through increased blood flow. The computer can also spatially determine where for example the pO₂ level has increased to the desired level and where it has not. The direction and focus of the radiotherapy and/or chemotherapy can then be altered so as to target those areas where the treatment will be effective, without applying the toxic treatments to regions which will not benefit from that treatment.

The energy unit 2 can be mounted on a robotic arm 4, manually, electronically, hydraulically and/or pneumatically controlled. In the case of automatic control, the information about the desired location where the energy is to be focused, will be sufficient to carry out a predetermined treatment programme. For example, for a relatively small tumour or thrombus, a single focal point may be used so that the energy interacts with a therapeutic agent (possibly an encapsulated agent) at that point. Alternatively, if a larger region of interest is mapped, the control means may cause a series of (ultrasound or EM) transmissions at different points throughout the region, following a predetermined pattern. The control unit 5 preferably also has the capability of optimizing the position of the therapeutic unit with respect to minimizing attenuation due to energy losses caused by bone, certain organs (e.g. lungs) natural cavities within the patient, and the like. The means of optimizing the position can be based on empirical values in relation to position data, and/or input from diagnostic and/or therapeutic devices.

With regard to the energy transmitting unit 2, low, e.g., but not limited to, 20 kHz to 2 MHz, frequency ultrasound exposure can be used in combination with liposomally encapsulated chemotherapeutic agents. Such low frequency ultrasound treatment can be non-hyperthermic, but can significantly increase the effect of liposomally encapsulated cytostatic drugs on tumor growth. Alternatively high, e.g., but not limited to, 1 MHz to 5 MHz, frequency ultrasound exposure can be applied to the region of interest to induce hyperthermia. A transducer unit can provide both low and high frequency ultrasound, simultaneously, concurrently or in sequence. The lower frequencies predominantly induce cavitation, while the higher frequencies are for inducing hyperthermia.

A system and methods have been provided for cancer and thrombi treatments in which hyperthermia is induced in an initial phase and cavitation and/or drug release are induced in a subsequent phase in a region of interest in a human or animal body. The system includes an energy transmitter having a variable intensity and/or a variable frequency; and a control unit arranged to control the energy transmitter to operate in at least two different modes. In one embodiment, the first mode is a mode of operation having a mechanical index below a threshold level for cavitation; and the second mode is a mode of operation having a mechanical index above a threshold level for cavitation. In another embodiment, the first mode induces hyperthermia below a temperature threshold for releasing an encapsulated agent and the second mode induces hyperthermia above the temperature threshold. The initial hyperthermia treatment can enhance the effect of subsequent treatments.

The present invention is not limited to the described apparatus, system or algorithms, thus all devices that are functionally equivalent are included by the scope of the invention. Modifications of the patent claims are within the scope of the invention.

Drawings and figures are to be interpreted illustratively and not in a limiting context. It is further presupposed that all the claims shall be interpreted to cover all generic and specific characteristics of the invention which are described, and that all aspects related to the invention, no matter the specific use of language, shall be included. Thus the stated references have to be interpreted to be included as part of this invention's basis, methodology, mode of operation and apparatus or system.

### APPENDIX

### Ultrasound Heating of Tissue And Cracking of Drug Carrying Nanoparticles

### Cracking

The fractional cracking of particles is given by the expression

$$\frac{n ( t )}{{N}_{0}} = 1 = {e}^{- t / {T}_{c} ( MI )} \qquad ( 1 )$$

where n(t) is the number of cracked particles and N₀ is the total number of particles (possibly per unit volume).

The Mechanical Index is given by:

$$MI = \frac{{P}_{neg}}{\sqrt{f}} \qquad ( 2 )$$

The cracking time constant is given by:

$${T}_{C} ( MI ) = \{ \begin{matrix}∞ for MI < {MI}_{0} \\ decrease monotonously for MI > {MI}_{0}\end{matrix} \qquad ( 3 )$$

### Power Delivered To Tissue

We operate with a Mechanical Index, MIₕ<MI₀ for heating. The pressure amplitude is then Pₕ=MIₕ\sqrt{ƒ} at the location of treatment. The delivered power per unit volume is

$$\begin{matrix}{W}_{h} = \frac{{P}_{h}^{2}}{2 {Z}_{0}} {μ}_{0} f = \frac{{MI}_{h}^{2}}{2 {Z}_{0}} {μ}_{0} {f}^{2} [ W \text{/} {m}^{3} ] absorbed power per unit volume & ( 4 ) \\ {I}_{h} = \frac{{P}_{h}^{2}}{2 {Z}_{0}} [ W \text{/} {m}^{2} ] acoustic radiation intensity & ( 5 )\end{matrix}$$

where Z₀≈1.6·10⁶ kg/m²s is the acoustic characteristic impedance of the tissue, and μ=μ₀ƒ is the power absorption coefficient of the ultraosund. We hence see that the delivered power for a given MI is ˜f². This suggests the use of a very high frequency for the treatment.

However, power absorption also influences the frequency selection in relation to the depth, z, of the treatment. The ultrasound frequency is due to absorption attenuated by the factor

e^{-μ}^{₀}^{ƒz} (6)

The treatment beam should also be focused at z, which gives a set of conditions for choosing the frequency that gives the best heat deposition at a given depth.

### Treatment Strategy

The above analysis indicates that for the heating as high an ultrasound frequency as possible should be used, taking increased ultrasound attenuation with frequency due to absorption and the beam focusing into account.

To achieve particle cracking, it is preferred to advantageously drop the frequency to increase the MI without dramatic increase in heating ( [symbol] : MIₕ²). If it is desired to maintain the heat delivery during the cracking, we obtain the requirement

$${MI}_{h}^{2} {f}_{h}^{2} = {MI}_{c}^{2} {f}_{c}^{2} \\ {f}_{c} = \frac{{MI}_{h}}{{MI}_{c}} {f}_{h} \qquad ( 7 )$$

where the subscript c indicates cracking and the subscript h indicates heating.

### EXAMPLE

With a relative bandwidth of 60% for a single band power transducer and with a center frequency f₀ we get

$${f}_{c} = 0.7 {f}_{0} \\ {f}_{h} = 1.3 {f}_{0} \\ {MI}_{c} = \frac{{f}_{h}}{{f}_{c}} {MI}_{h} = 1.86 {MI}_{h} \qquad ( 8 )$$

Alternatively, with a dual band power transducer there is a larger freedom to choose f_{c} and fₕ and hence also MI_{c} and MIₕ. The general rule is that it is desired to keep MIₕ as low as possible during the heating period to avoid cracking of the particles. This requires as high fₕ as possible. Similarly it is desired to keep MI_{c} as high as possible to get as fast cracking as possible (short T_{c}). With single band transducers, one might therefore accept potential over-heating during the cracking phase, while with dual band transducers there is a much larger freedom in selecting the parameters.

1.-40. (canceled)

41. A system for inducing hyperthermia and cavitation in a region of interest in a human or animal body comprising:
an energy transmitter having a variable frequency; and
a control unit arranged to control the energy transmitter to operate in at least two different modes,
wherein a first mode is a mode of operation having a mechanical index below a threshold level for cavitation;
wherein a second mode is a mode of operation having a mechanical index above a threshold level for cavitation,
wherein the ultrasound transmitter is a single frequency band ultrasound transmitter, and
wherein in the first mode of operation the ultrasound transmitter is arranged to operate at a high frequency above the centre frequency of the frequency band and wherein in the second mode of operation the ultrasound transmitter is arranged to operate at a low frequency below the centre frequency of the frequency band.

42. A system as claimed in claim 41, further comprising a monitoring unit arranged to monitor at least one parameter related to oxygenation in the region of interest.

43. A system as claimed in claim 42, wherein the monitoring unit is arranged to monitor the or each parameter spatially in and around the region of interest.

44. A system as claimed in claim 42, wherein the monitoring unit is an MRI unit arranged to monitor at least one of partial oxygen pressure, partial carbon dioxide pressure, acidity and temperature in the region of interest.

45. A system as claimed in claim 42, wherein the monitoring unit is arranged to supply data to the control unit and wherein the control unit is arranged to switch the energy transmitter from the first mode to the second mode based on said data.

46. A system as claimed in claim 42, wherein the control unit is arranged to switch the energy transmitter from the first mode to the second mode when the data indicates that the oxygenation of the region of interest has reached a threshold level.

47. A system as claimed in claim 42, wherein the monitoring unit is further arranged to monitor cavitation levels within the region of interest.

48. A system as claimed in claim 41, wherein the ultrasound transmitter comprises a single transducer or an array of transducers.

49. A system as claimed in claim 41, wherein the ultrasound transmitter comprises a high intensity focused ultrasound transmitter.

50. A system as claimed in claim 41, wherein the low frequency is up to 30% lower than the centre frequency and wherein the high frequency is up to 30% higher than the centre frequency.

51. A system as claimed in claim 41, wherein the low frequency is at least 5% lower than the centre frequency and wherein the high frequency is at least 5% higher than the centre frequency.

52. A system as claimed in claim 41, wherein the first mode of operation is suitable for inducing hyperthermia in the region of interest, but is not suitable for inducing cavitation in the region of interest.

53. A system as claimed in claim 41, wherein the second mode of operation is suitable for releasing an encapsulated therapeutic agent.

54. A system as claimed in claim 41, wherein the transmitter is further arranged to operate in a third mode of operation having a mechanical index above the threshold level for cavitation and being suitable for inducing cavitation of microbubbles.

55. A system for inducing hyperthermia and encapsulated agent release in a region of interest in a human or animal body comprising:
an energy transmitter having a variable intensity and/or a variable frequency; and
a control unit arranged to control the energy transmitter to operate in at least two different modes,
wherein a first mode is a mode of operation having an energy level below a threshold temperature level; and
wherein a second mode is a mode of operation having an energy level above a threshold temperature level.

56. A system as claimed in claim 55, further comprising a monitoring unit arranged to monitor at least one parameter related to oxygenation in the region of interest.

57. A system as claimed in claim 56, wherein the monitoring unit is arranged to monitor the or each parameter spatially in and around the region of interest.

58. A system as claimed in claim 56, wherein the monitoring unit is an MRI unit arranged to monitor at least one of partial oxygen pressure, partial carbon dioxide pressure, acidity and temperature in the region of interest.

59. A system as claimed in claim 56, wherein the monitoring unit is arranged to supply data to the control unit and wherein the control unit is arranged to switch the energy transmitter from the first mode to the second mode based on said data.

60. A system as claimed in claim 56, wherein the control unit is arranged to switch the energy transmitter from the first mode to the second mode when the data indicates that the oxygenation of the region of interest has reached a threshold level.

61. A system as claimed in claim 56, wherein the monitoring unit is further arranged to monitor cavitation levels within the region of interest.

62. A system as claimed in claim 56, wherein the energy transmitter comprises an electromagnetic energy transmitter.

63. A system as claimed in claim 62, wherein the electromagnetic energy transmitter is arranged to operate at a frequency between 100 MHz and 4 GHz.

64. A system as claimed in claim 55, wherein the energy transmitter comprises an ultrasound transmitter.

65. A system as claimed in claim 55, wherein the first mode of operation is suitable for inducing hyperthermia, but is not suitable for releasing an encapsulated agent, and wherein the second mode of operation is suitable for rupturing the vesicles of an encapsulated agent.

66. A system as claimed in claim 41, wherein the control unit is further arranged to switch the energy transmitter from the first mode to the second mode.

67. A method of inducing hyperthermia and cavitation in a region of interest in a human or animal body, comprising:
operating an energy transmitter which has a variable frequency in a first mode of operation having a mechanical index below a threshold level for cavitation, and
operating the energy transmitter in a second mode of operation having a mechanical index above a threshold level for cavitation,
wherein the ultrasound transmitter is a single frequency band ultrasound transmitter, and
wherein in the first mode of operation the ultrasound transmitter is operated at a high frequency above the centre frequency of the frequency band and wherein in the second mode of operation the ultrasound transmitter is operated at a low frequency below the centre frequency of the frequency band.

68. A method of inducing hyperthermia and encapsulated agent release in a region of interest in a human or animal body comprising:
operating an energy transmitter which has at least one of a variable intensity and a variable frequency in a first mode of operation having an energy level below a threshold temperature level; and
operating the energy transmitter in a second mode of operation having an energy level above a threshold temperature level.
